(19) United States
(12) Patent Application Publication
(10) Pub. No.: US 2025/0204845 A1
(43) Pub. Date: Jun. 26, 2025

(54) STYLUS DERIVED METRICS TO DETECT CENTRAL AND PERIPHERAL NERVOUS SYSTEM ATTRIBUTES

(71) Applicant: Linus Health, Inc., Boston, MA (US)

(72) Inventors: David Bates, Boston, MA (US); Alvaro Pascual-Leone, Boston, MA (US); Sean Tobyne, Boston, MA (US); Joyce Gomes-Osman, Boston, MA (US); John Showalter, Boston, MA (US); Ali Jannati, Boston, MA (US); Russell Banks, Boston, MA (US); Marissa Ciesla, Boston, MA (US); Karl Thompson, Cambridge, MA (US); Jeff Pobst, Boston, MA (US)

(21) Appl. No.: 18/897,839

(22) Filed: Sep. 26, 2024

Related U.S. Application Data
(60) Provisional application No. 63/612,596, filed on Dec. 20, 2023.

(51) Int. Cl. A61B 5/00; A61B 5/11

(52) U.S. Cl. CPC A61B 5/4088; A61B 5/1101; A61B 5/6897; A61B 5/7225; A61B 5/7264

(57) ABSTRACT

Systems and methods for using stylus-derived metrics to determine a cognitive impairment status. A method comprises receiving a collection of multimodal data of a user interaction with a computing device; processing the collection of multimodal data according to each modality within the collection of multimodal data; deriving one or more first order features from the processed collection of multimodal data; deriving one or more second order features from the processed collection of multimodal data; creating a patient data model based on the first and second order features; and applying a machine learning model to the patient data model to determine a cognitive impairment status.

### CROSS-REFERENCE TO RELATED APPLICATIONS

This application claims the benefit of U.S. Provisional Application No. 63/612,596, filed Dec. 20, 2023, which is hereby incorporated by reference in its entirety.

### TECHNICAL FIELD

The disclosure is generally directed to detecting cognitive impairment, and in particular, to detection of cognitive impairment through use of Bluetooth stylus metrics.

### BACKGROUND OF THE DISCLOSURE

Traditional cognitive impairment tests such as the MMSE, MoCA, and Mini-cog do not demonstrate the capacity to detect subtle issues of the central nervous system (CNS). Most of these tests were created to detect CNS impairment at a stage where a considerable number and the severity of symptoms have already developed. At this late stage, however, many common interventions are no longer viable due to the advanced nature of the impairment. Traditional CNS assessments involve clock test drawing abilities which are typically done using paper and pencil, require subjective judgment from personnel with specialized training, and can take up to 15 minutes to complete while also only looking at the final product drawing and not the process. Recent digital implementations of graphomotor functions that look at the process of drawing a clock have shown increased sensitivity to impairment at an earlier stage in the time course of disease. For example, complaints, symptoms, and disability of the central and peripheral nervous system (PNS) can be identified through subtle changes in drawing praxis (the ability to conceptualize, plan, and organize a task in order to successfully execute a motor skill from beginning to end) which is subserved directly by simple and complex motor skills but also indirectly by neurocognitive mechanisms of executive function and cognitive control. Additional time and subsequently resources are required for manually scoring such tests and deriving clinical insights.

Accordingly, there is a need for an objective, efficient, automated metric to identify, detect and classify early nervous system dysfunction in a healthcare setting.

### SUMMARY

The present disclosure is directed towards systems and methods for detecting cognitive impairment through stylus-derived metrics.

In one embodiment, a method comprises receiving a collection of multimodal data of a user interaction with a computing device; processing the collection of multimodal data according to each modality within the collection of multimodal data; deriving one or more first order features from the processed collection of multimodal data; deriving one or more second order features from the processed collection of multimodal data; creating a patient data model based on the first and second order features; and applying a machine learning model to the patient data model to determine a cognitive impairment status.

In some embodiments, the collection of multimodal data comprises data collected from one or more of a touchscreen, a stylus, a webcam, and/or a microphone.

In some embodiments, the first order features comprises one or more of a stroke-based drawing feature, a time-based drawing feature, an eye tracking feature, a sentiment feature, a stylus orientation feature, a stylus force strength feature, a speech content feature, and/or a speech aural qualities feature.

In some embodiments, the stylus orientation feature comprises a measurement of one or more of azimuth, altitude, temporal dynamics, and/or an ink distance.

In some embodiments, the stylus force strength feature comprises a measurement of pressure on the stylus.

In some embodiments, the collection of multimodal data is collected from a patient during a clinical assessment.

In some embodiments, the clinical assessment comprises a stylus component.

In some embodiments, the machine learning model comprises an artificial neural network.

In some embodiments, applying the machine learning model further comprises interpolating one or more stylus position from the processed collection of multimodal data at predetermined points in time; filtering the processed collection of multimodal data with both a low-pass and band-pass filter to determine an impulse response; and estimating a velocity and an acceleration of the stylus at the predetermined points in time.

In some embodiments, the method further comprises measuring a tremor in the stylus by: measuring total energy associated with the velocity and acceleration to determine one or more energy norms.

In some embodiments, the one or more energy norms include one or more of an acceleration magnitude, a perpendicular acceleration motion, and/or a parallel acceleration motion.

In some embodiments, the machine learning model is trained by comparing a tremor measured in a patient diagnosed with Essential Tremor with a tremor measured in a healthy patient.

In some embodiments, the method further comprises outputting an individual patient cognitive status.

In some embodiments, the method further comprises outputting a group level cognitive status, wherein the group comprises the user and each member having a common attribute.

In some embodiments, the method further comprises designating a class designation of cognitive impairment.

In some embodiments, the collection of multimodal data further comprises one or more questionnaire assessments or electronic health records, wherein, determining the second order feature uses data embedded in the one or more questionnaire assessments or electronic health records

In some embodiments, the method further comprises tracking the cognitive impairment status over time.

In some embodiments, the method further comprises detecting changes in the cognitive impairment status based on said tracking.

In some embodiments, a system comprises at least one input device; a computing node coupled to the at least one input device and comprising a computer readable storage medium having program instructions embodied therewith, the program instructions executable by a processor of the computing node to cause the processor to perform a method comprising: receiving a collection of multimodal data of a user interaction with the at least one input device; processing the collection of multimodal data according to each modality within the collection of multimodal data; deriving one or more first order features from the processed collection of multimodal data; deriving one or more second order features from the processed collection of multimodal data; creating a patient data model based on the first and second order features; and applying a machine learning model to the patient data model to determine a cognitive impairment status.

In some embodiments, a computer program product for determining a cognitive impairment status comprises a computer readable storage medium having program instructions embodied therewith, the program instructions executable by a processor to cause the processor to perform a method comprising: receiving a collection of multimodal data of a user interaction with a computing device; processing the collection of multimodal data according to each modality within the collection of multimodal data; deriving one or more first order features from the processed collection of multimodal data; deriving one or more second order features from the processed collection of multimodal data; creating a patient data model based on the first and second order features; and applying a machine learning model to the patient data model to determine a cognitive impairment status.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is an overview of a system for determining cognitive impairment status, as described by the techniques presented herein.

FIG. 2 is an illustration of stylus measurements, as described by the techniques presented herein.

FIG. 3 is a flowchart of an exemplary method for determining cognitive impairment status, as described by the techniques presented herein.

FIG. 4 is schematic view of an exemplary computing node.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Embodiments of the present disclosure correlate stylus-derived metrics (such as pressure, azimuth, altitude, temporal dynamics, and distance) obtained during a digital cognitive assessment (e.g., clock drawing test, trail-making test, etc.) to identify cognitive, motor, and mental health disorders. These metrics have singularly been used to aid in the diagnosis of some nervous system disorders such as dementia, concussion/traumatic brain injury, Parkinsonism, and Multiple Sclerosis. Additionally, embodiments of the present disclosure capture ideation, motor planning, execution, feedback, and adaptation of stylus-based praxis analysis (e.g., think time, drawing time, latency, sequence, etc.) to aid in the diagnosis of various nervous system disorders (e.g., dementia, concussion/traumatic brain injury, Parkinsonism, Multiple Sclerosis, etc.). The stylus-based praxis analysis may further enable physicians to distinguish subtypes, and the pathology (e.g., amyloid β, tau, etc.) of these disorders. Further, these stylus-based praxis metrics provide additional information over time regarding the change in drawing patterns which are indicative of cognitive, motor, and mental health decline.

Some embodiments of the present disclosure correlate the combination of stylus-derived praxis metrics with central nervous system (CNS) disorders by capturing ideation, motor planning, execution, and feedback and adaptation features.

Some embodiments of the present disclosure use individual or combined metrics such as grip strength, pressure, azimuth, altitude, temporal, and distance derived from the Bluetooth stylus to identify or classify cognitive, motor, functional, and mental health disorders. For example, stylus derived graphomotor features may be combined with speech, voice, and subjective lifestyle information and data to provide a memory impairment probability score for individuals testing on a platform.

Some embodiments of the present disclosure are used for the detection of complaints, symptoms, and disability of the CNS and peripheral nervous system (PNS) in a healthcare setting and provide clinical and patient-specific recommendations based on the metrics derived from the Bluetooth pencil.

Some embodiments of the present disclosure combine subjective (such as metrics from questionnaires) and objective features (such as metrics from a Bluetooth stylus) of a patient's CNS and PNS health.

Some embodiments of the present disclosure correlate pressure, azimuth, altitude, temporal, and distance metrics derived from the Bluetooth stylus to cognitive, motor, functional, and mental health disorders. These features are used to specifically aid in the classification of brain dysfunction like cognitive impairment and dementia subtypes such as mild cognitive impairment (MCI), Alzheimer's disease (AD), vascular dementia (VaD) and movement/motor disorders (e.g., Parkinsonism, dystonia, dyspraxia and tremor), as well as concussion/TBI, and Multiple Sclerosis.

Leveraging multiplexing algorithms (detailed below), which include stylus-derived grip strength, pressure sensing, angles and degrees of movement, complaints, symptoms, and disability of the CNS and PNS are concurrently assessed in attention, executive function, language, visuospatial reasoning, motor skills, verbal memory, and information processing domains. Subtle and clinically meaningful changes are detected over time in cognitive, motor, mental health, and functional performance either at the individual or group level by incorporating stylus-based praxis analysis and composite scores to these algorithms. Longitudinal tracking can be used to monitor an individual's cognitive and motor health over time to observe potential longitudinal change and guide clinical interventions. Additionally, the longitudinal data from all participating individuals will be used to understand how the collected Bluetooth stylus metrics correlate to cognitive and motor changes over time. Longitudinal data collection also allows for more robust statistical analysis and improves the clinical meaningfulness of results.

Embodiments of the present disclosure include systems and methods for graphomotor process metrics derived from a Bluetooth stylus, tablet and OS output to develop and train machine-learning models to classify or predict cognitive impairment (CI; combining MCI and mild dementia likely due to AD), PET Aβ± status using cognitive testing and blood biomarkers (BBM), motor impairment, mental health disorders, and functional impairment.

### Description of the Algorithm

Filters are first applied to the data obtained prior to applying them in the algorithm. Let x[n]=(x[n], y[n])′ be the column vector of interpolated pen positions for n=0, . . . , N−1. Because the filters may be odd-length and either symmetric (low-pass, bandpass) or anti-symmetric (derivative), it may be convenient to center all the filters at the origin. Let hL[n] be the impulse response of the low-pass filter with non-zero taps[−NL,NL], let hB[n] be the impulse response of the band-pass filter with taps[−NB,NB], and let hD[n] be the impulse response of the derivative filter with taps[−ND,ND]. It may be convenient, though not necessary, to have the low-pass and bandpass filter lengths be the same, where NL=NB. Then the smoothed, low-pass filtered position may be:

$${x}_{L} [ n ] = {\sum }_{k = - {N}_{L}}^{{N}_{L}} {h}_{L} [ k ] x [ n - k ] for n = {N}_{L} , … , N - 1 - {N}_{L}$$

The smoothed velocity estimates may be annotated:

$${v}_{L} [ n ] = {\sum }_{k = - {N}_{D}}^{{N}_{D}} {h}_{D} [ k ] {x}_{L} [ n - k ] for n = {N}_{D} + {N}_{L} , … , N - 1 - {N}_{D} - {N}_{L}$$

Bandpass filtering is applied to velocities vB [n]=(vBx[n], vBy[n])′ and accelerations aB [n]=(aBx[n], aBy[n]) may be derived as follows:

$${x}_{B} [ n ] = {\sum }_{k = - {N}_{B}}^{{N}_{B}} {h}_{B} [ k ] x [ n - k ] for n = {N}_{B} , … , N - 1 - {N}_{B}$$

$${v}_{B} [ n ] = {\sum }_{k = - {N}_{D}}^{{N}_{D}} {h}_{D} [ k ] {x}_{B} [ n - k ] for n = {N}_{D} + {N}_{B} , \text{} … , N - 1 - {N}_{D} - {N}_{B} {a}_{B} [ n ] = {\sum }_{k = - {N}_{D}}^{{N}_{D}} {h}_{D} [ k ] {x}_{B} [ n - k ] for n = \text{} 2 {N}_{D} + {N}_{B} , \text{} … , N - 1 - 2 {N}_{D} - {N}_{B}$$

The velocity in the direction of the pen motion is calculated by taking the inner product between the bandpass velocity with the direction of the low-pass filtered velocity. This can also be viewed as a projection operation, and it results in the signal vP[n]. The velocity perpendicular to pen motion may be calculated by taking the cross-product between the bandpass velocity with the direction of the low-pass filtered velocity. This gives signal v⊥[n].

$${v}_{P} [ n ] = \frac{1}{‖ {v}_{L [ n ]} ‖} ( {V}_{L x} [ n ] {v}_{B x} [ n ] + {V}_{L y} [ n ] {v}_{B y} [ n ] )$$

$$for$$

$$n = {N}_{D} + {N}_{B} , … , N - 1 - {N}_{D} - {N}_{B} {v}_{⊥} [ n ] = \frac{1}{‖ {v}_{L [ n ]} ‖} ( {V}_{L x} [ n ] {v}_{B y} [ n ] + {V}_{L y} [ n ] {v}_{B x} [ n ] )$$

Similarly, to compute the acceleration in the direction of pen motion, take the inner product of the bandpass acceleration with the direction of the low-pass filtered velocity, giving a_{P}[n]. To compute the acceleration perpendicular to pen motion, take the cross-product between the bandpass acceleration with the direction of the low-pass filtered velocity, giving a⊥[n].

$${a}_{P} [ n ] = \frac{1}{‖ {v}_{L [ n ]} ‖} ( {V}_{L x} [ n ] {a}_{B x} [ n ] + {V}_{L y} [ n ] {a}_{B y} [ n ] )$$

$$for$$

$$n = 2 {N}_{D} + {N}_{B} , … , N - 1 - 2 {N}_{D} - {N}_{B} {a}_{⊥} [ n ] = \frac{1}{‖ {v}_{L [ n ]} ‖} ( {V}_{L x} [ n ] {a}_{B y} [ n ] + {V}_{L y} [ n ] {a}_{B x} [ n ] )$$

If the user were drawing a perfect circle at constant velocity, and the filtering was ignored, then vP[n] would be expected to be the pen velocity, and v⊥[n] would be zero. Acceleration would be expected to be perpendicular to the velocity and aligned in the radial direction when drawing a perfect circle so that aP[n] should be zero and a⊥[n]=vP 2[n]/Radius. Of course, even healthy subjects do not draw perfect circles.

### Tremor Quantification Features

The strength of the bandpass velocity and acceleration signals could be used to quantify the tremor in a number of ways. The system could measure the total energy in one or more of the bandpass filtered and/or parallel/perpendicular signals, different energy norms could be used (sum of squares, L1 norm, and so forth), higher order derivatives could be measured, windows could apply different weights to the center vs. the ends of the stroke in a fixed or adaptive manner, and so forth. As an example, some useful features that correlate well with tremor are:

$$PnAccLogEnergy = log ( \frac{1}{{K}_{A} {M}_{A}} {\sum }_{n} {‖ {a}_{B} [ n ] ‖}^{2} )$$

$$PnCurvLog Energy = log ( \frac{1}{{K}_{A} {M}_{A}} {\sum }_{n} {a}_{⊥}^{2} [ n ] )$$

$$PnAccParLog Energy = log ( \frac{1}{{K}_{A} {M}_{A}} \sum {a}_{p}^{2} [ n ] )$$

$$PnSpdLog Energy = log ( \frac{1}{{K}_{A} {M}_{A}} {\sum }_{n} {‖ {v}_{B} [ n ] ‖}^{2} )$$

$$PnSpdPerpLog Energy = log ( \frac{1}{{K}_{V} {M}_{V}} {\sum }_{n} {v}_{⊥}^{2} [ n ] )$$

$$PnSpdParLog Energy = log ( \frac{1}{{K}_{V} {M}_{V}} {\sum }_{n} {v}_{P}^{2} [ n ] )$$

Here PnAccLogEnergy is the energy (sum of squares) of the bandpass filtered acceleration magnitude, normalized by dividing by the number of terms in the sum, MA=N−4ND−2NB, and further normalized by a scaling factor, KA, that may compensate for drawing size, drawing time, overall speed, and so forth. The log( ) function may be used to make the distribution of feature values look more Gaussian. PnCurvLogEnergy is a similar feature that only sums the energy in the acceleration component perpendicular to pen motion. This can also be thought of as a measurement of the energy of the curvature of the stroke. PnAccParLogEnergy measures the energy in the acceleration component parallel to the pen motion. PnSpdLogEnergy is the energy (sum of squares) of the bandpass filtered velocity magnitude, normalized by dividing by the number of terms in the sum, MV=N−2ND−2NB, and further normalized by a scaling factor, KV, that may compensate for drawing size, drawing time, overall speed, and so forth. The log( ) function may be used to make the distribution of feature values look more Gaussian. PnSpdPerpLogEnergy is similar, but it sums the energy in the perpendicular bandpass velocity signal. PnSpdParLogEnergy is similar, but it sums the energy in the parallel bandpass velocity signal.

The normalizing factors may be important to achieving good results. For example, suppose the subject drew a clock that was doubled the height and width of the normalized drawing but still drew the clock in the same amount of time. All velocities and accelerations would double. On the other hand, if the diagram were drawn in the same size but in twice the time, the velocities would halve and the accelerations would drop by a quarter. In both of these examples, however, it may be desirable for the tremor feature value to remain unaltered. Compensating for these factors may be important because individuals affected by movement disorders may draw relatively small diagrams, moving the pen carefully and slowly.

One example technique is to normalize the bandpass velocity by dividing by the average velocity one would expect given the length of ink and the time required to draw the clock face.

$$[ n ] = {v}_{B} [ n ] / {v}_{a v g} where {v}_{a v g} = \frac{Length of Ink}{Drawing Time}$$

Doubling the size or doubling the time would then leave features depending on bandpass velocity unchanged. Similarly, bandpass acceleration may be normalized by dividing by the average acceleration:

$$[ n ] = {a}_{B} [ n ] / {a}_{a v g} where {v}_{a v g} = \frac{Length of Ink}{Drawing {Time}^{2}}$$

The preceding embodiments may underestimate the complexity of the normalization problem since speeding up and slowing down or drawing large or small may also change the frequency content of the tremor, which in turn may change how much energy passes through the bandpass filter and thereby change the feature. It may not be clear how the amplitude and frequency of the tremor changes when the subject tries to write faster or larger. In some embodiments, velocity and acceleration may be normalized by the same or similar factor, the square of the ratio of ink length to drawing time. For the features above, this may yield normalizing constants.

$${{K}_{A} = {K}_{V} = ( \frac{Length of Ink}{Drawing Time} )}^{2}$$

Similar strategies may use the size of the drawing, size of the bounding box, average radius, and so forth, instead of length of ink (sum of all pen strokes used in the drawing; i.e., ink time). Each of the features described above may increase with praxis abnormalities, and so may be viewed as tremor scores. Other variations of these features such as those discussed earlier can be devised that may also behave as tremor scores. In addition, multiple features may be combined in formulas or algorithms in order to build yet other tremor scores.

The digital clock test may have two separate clock drawings, the “command” where the individual draws the clock from memory, and the “copy” where the individual copies a pre-drawn clock. Tremor may be estimated using the two outline strokes separately, and scores may be combined from the two clock faces in the two clocks. Including more pen stroke data (such as data obtained when drawing additional details of the clock, e.g., numbers and hands) may improve the ability to distinguish between presence and absence of tremor. In addition, multiple features may be combined, for example a feature using velocity may be combined with another using acceleration, in an attempt to use whatever additional information might be available.

### Comparing Essential Tremor and Healthy Subjects

Subjects were selected who were diagnosed to have Essential Tremor (60 total, 36 males, age 65.6±11.8 years) or who were healthy controls (59 total, 14 males, age 55.1±12.5 years). They were administered Digital Clock Drawing tests, and the features on each of the command and copy clock face outlines were computed for these subjects. When the Digital Clock Test is used to screen for cognitive impairments, the Command and Copy clocks may have quite different characteristics, but they may be equally susceptible to tremor and may be treated equally. histograms were computed of each feature for the healthy and Essential Tremor populations, and ROC curves were computed by considering all possible thresholds. Effective measures for this data set included PnAccLogEnergy, the energy in the bandpass acceleration, PnSpdPerpLogEnergy, the energy in the bandpass velocity perpendicular to the low pass filtered velocity, and PnCurvLogEnergy, the energy in the component of bandpass acceleration perpendicular to the smoothed velocity (curvature).

In this test, these histograms were normalized by dividing by the number of clock faces so that the height of each bar represented a frequency of occurrence. In general, for all three scoring metrics, subjects with tremor have scores that were usually larger than scores of healthy subjects. In all three cases, there is some overlap in scores between the tails of the tremor and healthy populations. In general, though, these three metrics had comparable performance at separating the Essential Tremor and healthy populations.

To generate a classification of individual performance, a feature list like the one discussed above is chosen and compared with a threshold determined based on established neuropsychological evidence and norm-based thresholds. If the score is above or below that threshold in the direction of impairment, the subject is assigned the disordered label (for example, “Motor Impairment”, and if not, then the subject is assigned the “healthy” label. The probability of false positive (PF) is the fraction of subjects who have been diagnosed by medical professionals as healthy, but whose feature scores are impaired in the direction of impairment from the threshold. The probability of detection (PD) is the fraction of subjects who have been diagnosed by medical professionals as having tremor, and for whom the feature score is above threshold. The individual's cognitive classification may be built by calculating PF and PD for all possible thresholds and plotting these two statistics in a 2D graph. At one extreme with the threshold set to −∞ no patients are diagnosed as having tremor so PF=0 and PD=0. At the other extreme with the threshold set to +∞ all patients are diagnosed as having tremor, so PF=1 and PD=1. A perfect test would have a threshold such that PF=0 and PD=1.

A metric called Area Under the Curve (AUC) measures the area underneath the ROC curve. A perfect test would have AUC=1.0, while a test that had no discrimination ability at all would have AUC=0.5. Area Under the Curve (AUC) values from the data set presented can be found in Tables 1 and 2.

TABLE 1
AUC for different metrics, treating the
two drawings separately or combined.

| | Command and Copy | Command + Copy |
|---|---|---|
| Metric | separately | combined |
| PnAccLogEnergy | 0.970 | 0.977 |
| PnCurvLogEnergy | 0.977 | 0.987 |
| PnSpdPerpLogEnergy | 0.980 | 0.987 |

To describe what the feature values imply about visible tremor in the drawings, the healthy and Essential Tremor subjects had the following feature values:

TABLE 2
Values for Healthy and Essential Tremor Subjects

| Subject | PnAccLogEnergy | PnCurvLogEnergy | PnSpdPerpLogEnergy |
|---|---|---|---|
| Healthy Subject | 9.4 | 8.4 | 1.09 |
| Essential Tremor | 13.9 | 13.0 | 5.21 |

Other features from both drawings may be used to further improve the score. For example, on the data set disclosed above, if (PnCurvLogEnergy+PnSpedPerpLogEnergy)/2 is averaged over the two drawings and used as the score, it raises the AUC for this particular dataset to 0.99. However, in general, gains from combining these features may be limited because they may be highly correlated.

### Bluetooth Stylus Metrics

The Bluetooth stylus metrics, including pressure/force, azimuth, altitude, distance, temporal dynamics (drawing time), stroke count, and grip and pinch strength all have distinct signals/strengths that make them powerful for predicting cognitive outcomes of interest. The features included with Pencil metrics to determine an individual's potential cognitive health status are shown below in Table 3.

TABLE 3
Bluetooth stylus metrics

| Stylus-Derived | |
|---|---|
| Metrics | Definition |
| Pressure/Force | The force of the touch, where a value of 1 represents the force of an |
| | average touch, a predetermined benchmark set by the stylus system. |
| | The force reported is measured along the axis of the pencil and is |
| | included into a deep learning model that produces a feature prediction |
| | for each patient. This may be used to predict pinch grip strength, |
| | mental health status, functional impairment, motor, and cognitive |
| | ability. Pressure, in particular the pressure maximums of each stroke, |
| | is strongly user-specific and has interesting variation within a |
| | user/section that may make it an important indicator. |
| Azimuth | Azimuth is a measure of the direction in which the stylus (in |
| | particular the stylus cap) is pointing in radians relative to the |
| | plane of the screen. From the docs “With the tip of the stylus |
| | touching the screen, the value of this property is 0 radians when |
| | the cap end of the stylus (that is, the end opposite of the tip) |
| | points along the positive x axis of the device's screen. The |
| | azimuth angle increases as the user swings the cap end of the |
| | stylus in a clockwise direction around the tip.” |
| Altitude | Altitude is a measure of the elevation of the stylus above the plane of |
| | the screen in radians. From the docs. A value of 0 radians indicates |
| | that the stylus is parallel to the surface, which increases as the user |
| | lifts the cap-end from the surface and is included into a deep learning |
| | model that produces a feature prediction for each patient. This may be |
| | used to predict mental health status, functional impairment, motor, |
| | and cognitive ability. |
| Distance | Two measures of distance include: |
| | d = distance that stylus writing tip traveled across tablet screen |
| | D = distance non-writing end of stylus traveled while writing on the |
| | tablet screen |
| Temporal | The amount of time that the stylus is in contact with the device |
| Dynamics | surface and the per stroke drawing time (total drawing time/stroke |
| (Drawing Time) | count). |
| Stroke Count | The number of individual pen strokes produced in the drawing of |
| | either the command or copy clock, with each stroke delineated by |
| | each stylus point-end lift from the tablet surface and incorporated into |
| | a deep learning model that produces a feature prediction for each |
| | patient. This may be used to predict mental health status, functional |
| | impairment, motor, and cognitive ability. |
| Grip and Pinch | Grip strength is a measure of muscular strength or the maximum |
| Strength | force/tension generated by one's hand and fingers. Key for tasks |
| | requiring fine motor skills |
| | This may be used to predict mental health status, functional |
| | impairment, motor, and cognitive ability. |

Digital assessments, such as The Digital Clock and Recall (DCR; Linus Health Inc) including the DCTclock™, immediate, and delayed recall tests; Anxiety1; Depression2,3; General Mental Health4; Spiral Tracing; Trail Making Test (A and B); and functional independence require use of a Bluetooth stylus (e.g., Apple Pencil) and tablet in behaviors such as drawing and/or tapping to detect signs of impairment. The metrics obtained from the Bluetooth stylus during these assessments are used to classify nervous system dysfunction. For example, a random-forest classifier was used for DCR models, which included metrics of Command and Copy Clock completion, immediate and delayed recall tests, and participant's age. Additional features included those extracted by the DCTclock algorithm, like our novel clock-drawing features, and temporal and spatial features (shown in Table 4) derived from the process and use of a Bluetooth stylus (e.g., altitude, azimuth, pressure).

Composite scores of the DCTclock are generated by passing specific key features into four separate logistic regression classifiers trained to differentiate between cognitively unimpaired and cognitive impaired subjects. Thus, composite scales are weighted combinations of several underlying key features. The coefficients are hard-coded within the DCTclock algorithm codebase. All features, composite scales and the final DCTclock score are then standardized by age-category means and standard deviations for appropriate values. Mean and standard deviation values are hardcoded into the DCTclock algorithm code. Below are the features and definitions associated with each Bluetooth stylus-derived objective measure used in our cognitive classification models in the DCR.

TABLE 4
DCR Features derived from the Bluetooth stylus

| Clock features derived from stylist metrics | Definition |
|---|---|
| Delayed Recall accuracy score | The accuracy count of the 3-word recall test |
| | displayed in a score of 0-3. |
| DCTclock Spatial Reasoning composite score | The spatial abilities demonstrated during the process |
| | of drawing a copy of the displayed clock. This |
| | feature considers metrics pertaining to the geometric |
| | properties of the drawing including the circularity of |
| | the clock circle, placement of clock components, and |
| | drawing placement on the page. |
| DCTclock Oscillatory Motion | A measure of how much the motion of the pen |
| | deviates from a smooth pen motion during the |
| | drawing process of the copying of the clock. |
| Functional Impairment Questionnaire | Activities of daily living (ADL), instrumental |
| | activities of daily living (iADL), and frailty |
| | questionnaires. |
| Temporal Speech Features: | Speech timing features related to speech fluency and |
| Long and short pause count between | the general speed of the verbal responses and |
| words | reproductions of the words. |
| Time to first word | |
| Hesitations | |
| Pause time (%) | |
| Acoustic Voice Features: | Acoustic voice features associated with the loudness, |
| Frequency: average, range, StDev., jitter | pitch, and overall quality of the produced speech. |
| Volume: average, range, StDev., shimmer | |
| Spectral features: Cepstral peak | |
| prominence | |
| Voice time (%) | |
| Speech and Language Production Features: | Speech and production features related to language, |
| Filler words and sounds | words and sounds that are traditionally associated |
| % of filler words to relevant content | with the presence of cognitive impairment. |
| words | |
| Semantic language | |
| Relevant content units | |
| Eye Gaze and Sentiment Features: | Gaze tracking features produced from video |
| Spatial distribution of eye movements | recordings of the eye movements of the patient |
| Frequency and severity of fluctuations in | undertaking the DCR (DCTClock test + 3 word |
| eye movement | recall test). A deep learning model analyzes the |
| Fixation frequency and average duration | video recording frame-by-frame and produces a gaze |
| Locating and response time in Copy | and sentiment prediction for each frame. Post- |
| Clock | processing algorithms analyze the gaze predictions |
| Number of switches between prompt and | and produce the given gaze tracking features for the |
| drawing areas in Copy Clock | patient. |
| Percentage time looking off screen | |
| Average gaze distance ahead of stylus | |
| Stationary gaze entropy / transition gaze | |
| entropy | |
| Hemispheric visual neglect | |
| Blink rate | |
| Linus Life and Health Questionnaire: | The Life and Health Questionnaire (LHQ) is a brief |
| Subjective/ Patient-reported | digital survey that captures key lifestyle and health |
| Depression/Anxiety | factors for cognitive impairment/dementia. These |
| Frailty | questions have been demonstrated to significantly |
| Sleep | predict functional status both in isolation and also |
| Nutrition | when combined with other metrics listed above. |
| Physical Activity | |
| Social engagement | |
| Cognitive stimulation | |
| Life Satisfaction/Purpose | |
| Depression Questionnaires | Geriatric Depression Scale (GDS) |
| Anxiety Questionnaires | General Anxiety Questionnaire (GAD-7) |
| General Mental Health | Patient Health Questionnaire-8 |

The metrics and subsequent CNS disorder classification can be applied in health care settings. For example, based on the metrics obtained, cognitive, motor, mental health, and functional assessment scores and next-step recommendations for patient care are populated. Along with this, patient-specific recommendations based on assessment or predictive classification of their cognitive, motor, mental health, and functional performance along with personalized lifestyle recommendations are populated. Embodiments of the present disclosure reduce assessment administration time by more than 10 minutes and subsequently, patient visit times and aids providers in clinical next step and follow-up care.

Embodiments of the present disclosure include a method to detect subtle and clinically meaningful changes over time in an individual's cognitive, motor, mental health, and functional performance through the analysis of several metrics derived from a Bluetooth stylus. Additionally, these models have the ability to detect group level changes in cognitive ability focusing on various aspects like age, race, education, and other demographic features.

The metrics derived from the Bluetooth stylus can be used for the detection of complaints, symptoms, and disability of the central and peripheral nervous system such as cognitive disease, motor impairment, mental health disorders and functional impairment in a healthcare setting.

Current practices have incorporated the use of tools like Microsoft Surface and Pen, Logitech stylus, and others to monitor/evaluate motor drawing behaviors. These tools have also been used to elevate and enhance clinical rehabilitation sessions. In these instances, the stylus is used as a measure of engagement in rehabilitation and not a means of predicting pathology, classifying cognitive performance/impairment, or understanding deficits in task patterns. Additionally, other practices have not used stylus-derived features to detect cognitive impairment, functional impairment, motor impairment, or mental health disorders for the purposes of next-step recommendations and clinical decision support (CDS) functionality that aids clinicians in providing patient care.

Traditional cognitive assessments alone cannot accurately assess or predict both the cognitive consequences and pathological substrates of Alzheimer's disease, mainly because this is outside of the scope of their design. The majority were designed to capture only those cases with deficits severe enough to manifest on a non-sensitive assessment. Further, traditional cognitive assessments are paper and pencil based and are unable to digitally process features potentially associated with disorders of the CNS. In contrast, the cognitive and functional assessment and Bluetooth stylus derived graphomotor features achieved success in both the assessment and prediction of cognitive pathology. Digital cognitive assessments, such as the DCR, present opportunities for efficient integration into clinical practice for accurate and early identification of both cognitive impairment and AD biomarker status.

Incorporating the stylus-based praxis data to the algorithms is that compared to existing solutions, particularly those incorporating the use of wearable technology, such as applications deriving measures of postural stability and gait, a much more finite process of graphomotor control is examined in two different contexts: Command and Copy clock. In doing so, greater accuracy and sensitivity is achieved, and methods can further include the ability to determine class designation of cognitive impairments, wherein class designation may include a severity of the cognitive impairment.

Assessments that use process metrics of motor planning and execution as well as spatial and temporal features derived from a Bluetooth stylus include the DCR, Spiral Tracing, Trails, and functional assessments. The DCR analyzes an individual's performance on a combination of clock drawing and word recall tasks. The four components of the DCR are the Copy Clock, Command Clock, Immediate Recall, and Delayed Recall. The digital Copy and Command clocks are analyzed using a proprietary machine-learning algorithm, known as the DCTclock™ (Linus Health, Inc.). Embodiments of the present disclosure include a method to detect subtle and clinically meaningful changes in an individual's or a group-level cognitive and functional performance through the analysis of several metrics derived from a stylus-based praxis analysis.

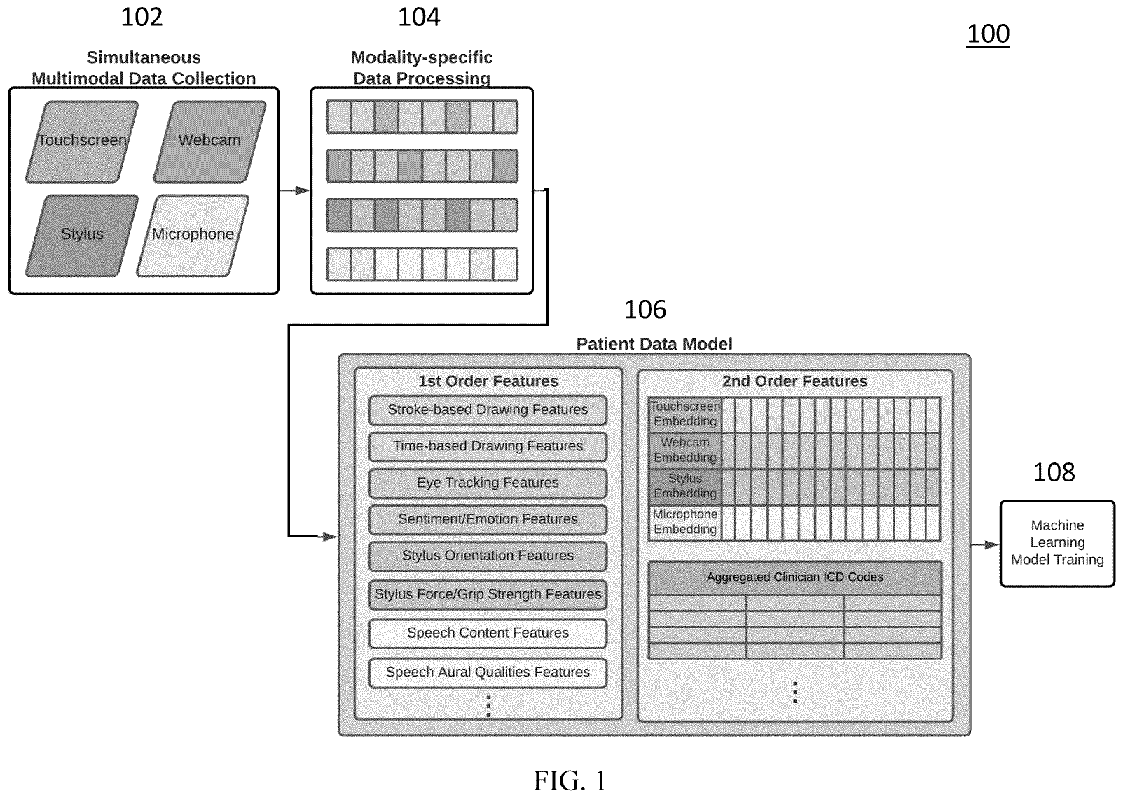

FIG. 1 depicts a general overview of a multiplexed approach 100, combining several elements of assessment completion to CNS/PNS and process assessment. The system works by recording data time series in step 102 using the tablet's native touchscreen (e.g., drawing strokes), paired stylus (e.g., Apple Pencil altitude, azimuth, pressure), native microphone (e.g., task-elicited or spontaneous speech, background noise), and/or native camera (e.g., eye blink rate, eye blink length) while the participant performs one of a set of predefined cognitive assessments. Each unimodal data stream undergoes modality-specific data processing (e.g., denoising, scaling, normalizing) in step 104 before 1st and 2nd order features are derived. Data time series are also embedded into a lower dimensional space (e.g., via an autoencoder) and combined with additional information gathered from questionnaires or electronic health records, for example, to construct a patient-specific ‘Data Model’ in step 106. Any elements of the Data Model are then used as inputs to an array of machine-learning models in step 108.

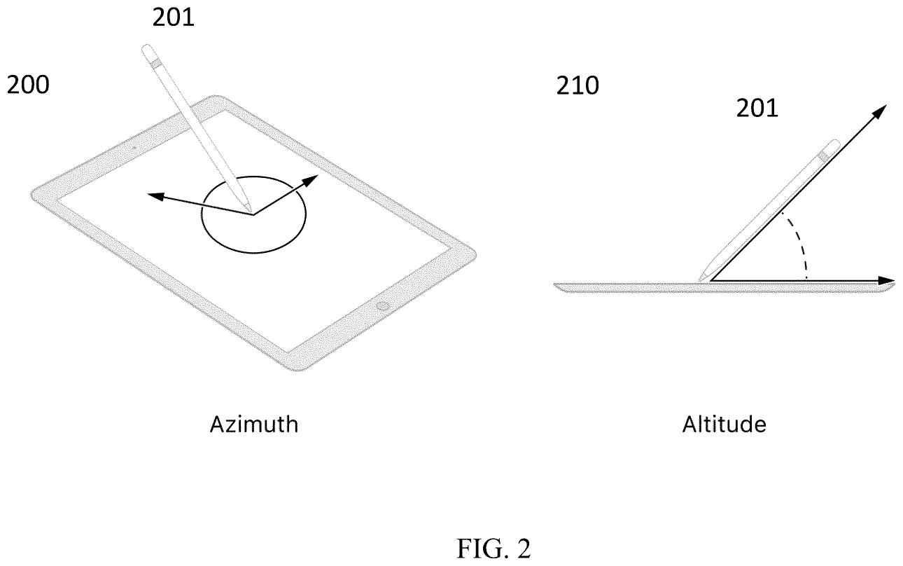

FIG. 2 is a visualization of Bluetooth stylus-derived features of azimuth and altitude contributing to the cognitive impairment classification system. In diagram 200, a stylus 201 is in use while the azimuth measurement is taken. In diagram 210, the stylus is in use while the altitude measurement is taken.

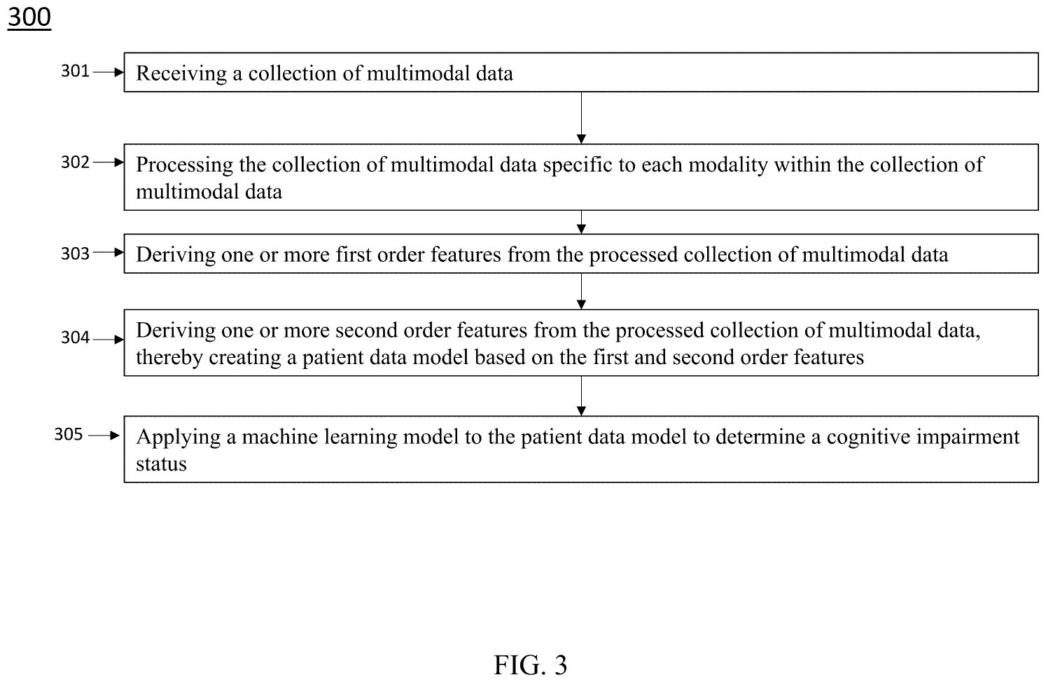

FIG. 3 is a flowchart of an exemplary method for determining a cognitive impairment status based on Bluetooth stylus derived metrics. In step 301, the method includes receiving a collection of multimodal data. In step 302, the method includes processing the collection of multimodal data specific to each modality within the collection of multimodal data. In step 303, the method includes deriving one or more first order features from the processed collection of multimodal data. In step 304, the method includes deriving one or more second order features from the processed collection of multimodal data, thereby creating a patient data model based on the first and second order features. An example of a first order feature include stroke-based drawing features and time-based drawing features from the touchscreen data, as well as eye tracking features and sentiment/emotion features from the webcam feature data. Additionally, stylus orientation features as well as stylus force/grip strength features data from the stylus may be considered first order features. Second order features may include, for example, include data embedded within each modality by which the patient provides a response. In step 305, the method includes applying a machine learning model to the patient data model to determine a cognitive impairment status.

Stylus features from 930 racially and ethnically diverse individuals were examined and used in a statistical model to predict physician-confirmed and/or neuropsychologically defined cognitive classifications of cognitively unimpaired, mild cognitive impairment, or probable dementia due to Alzheimer's disease per study protocol.

Participant individual performance and process on each of 3 cognitive assessments and status of 4 blood biomarkers alone, or their combinations, were used to classify cognitive impairment. Amyloid-beta status was also evaluated using the area under the receiver operating characteristic curve (AUC). Superiority, non-inferiority, or inferiority were established by comparing bootstrapped confidence intervals for classification accuracy with a 10% margin. 35% of participants were amyloid-beta positive on PET. Aβ42/40, pTau-181, and pTau-217 poorly classified cognitive impairment (AUCs: 0.63; 0.66; 0.72, respectively), but accurately classified amyloid-beta status (AUCs: 0.81; 0.78; 0.89, respectively). Digital Clock and Recall (DCR) accurately classified cognitive impairment (AUC=0.85) and amyloid-beta status (AUC=0.83). The DCR (including stylus-derived features) was superior or non-inferior to other cognitive tests for cognitive-impairment classification, and to Aβ42/40, pTau-181, and pTau-217 for amyloid-beta classification.

Features provided by the Bluetooth stylus are used to understand the cognitive health status of an individual using the Pencil to complete a variety of digital cognitive tasks incorporating stylus use. Composite scores are generated by passing specific key features into four separate logistic regression classifiers trained to differentiate between cognitively unimpaired and cognitive impaired subjects. Thus, composite scales are weighted combinations of several underlying key features. The coefficients are hard-coded within the DCTclock algorithm codebase. All features, composite scales and the final DCTclock score are then standardized by age-category means and standard deviations for appropriate values. Mean and standard deviation values are hardcoded into the DCTclock algorithm code.

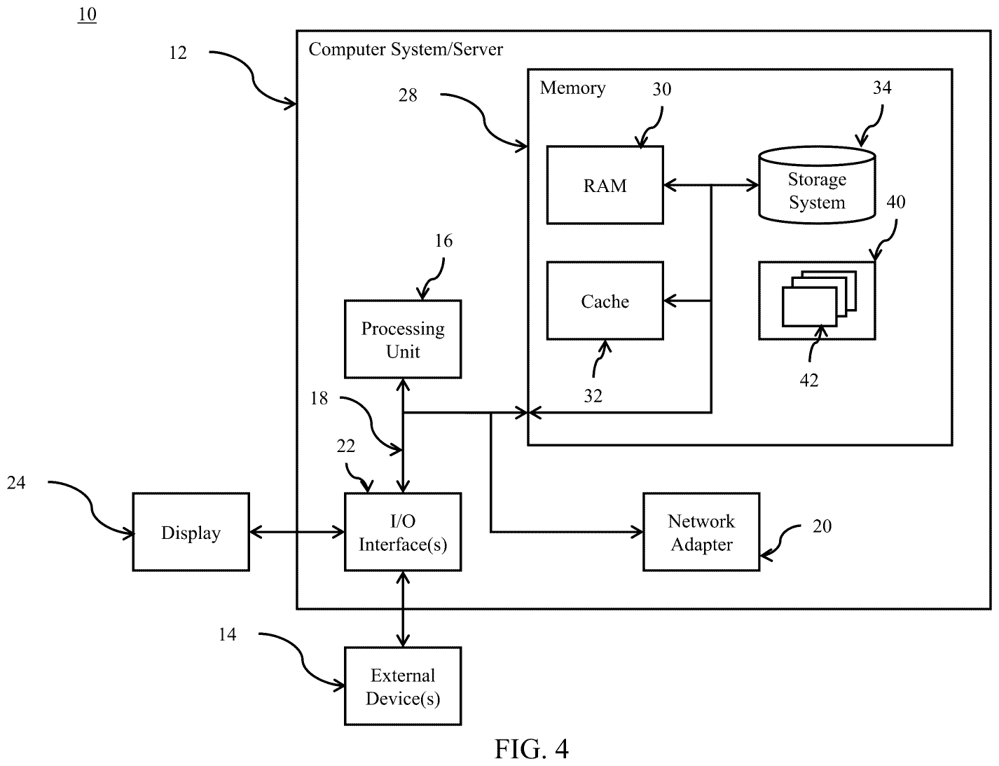

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving a collection of multimodal data of a user interaction with a computing device;
processing the collection of multimodal data according to each modality within the collection of multimodal data;
deriving one or more first order features from the processed collection of multimodal data;
deriving one or more second order features from the processed collection of multimodal data;
creating a patient data model based on the first and second order features; and
applying a machine learning model to the patient data model to determine a cognitive impairment status.

2. The method of claim 1, wherein the collection of multimodal data comprises data collected from one or more of a touchscreen, a stylus, a webcam, and/or a microphone.

3. The method of claim 1, wherein the first order features comprises one or more of a stroke-based drawing feature, a time-based drawing feature, an eye tracking feature, a sentiment feature, a stylus orientation feature, a stylus force strength feature, a speech content feature, and/or a speech aural qualities feature.

4. The method of claim 3, wherein the stylus orientation feature comprises a measurement of one or more of azimuth, altitude, temporal dynamics, and/or an ink distance.

5. The method of claim 3, wherein the stylus force strength feature comprises a measurement of pressure on the stylus.

6. The method of claim 1, wherein the collection of multimodal data is collected from a patient during a clinical assessment.

7. The method of claim 6, wherein the clinical assessment comprises a stylus component.

8. The method of claim 1, wherein the machine learning model comprises an artificial neural network.

9. The method of claim 1, wherein applying the machine learning model further comprises:
interpolating one or more stylus position from the processed collection of multimodal data at predetermined points in time;
filtering the processed collection of multimodal data with both a low-pass and band-pass filter to determine an impulse response; and
estimating a velocity and an acceleration of the stylus at the predetermined points in time.

10. The method of claim 9, further comprising measuring a tremor in the stylus by:
measuring total energy associated with the velocity and acceleration to determine one or more energy norms.

11. The method of claim 10, wherein the one or more energy norms include one or more of an acceleration magnitude, a perpendicular acceleration motion, and/or a parallel acceleration motion.

12. The method of claim 10, wherein the machine learning model is trained by comparing a tremor measured in a patient diagnosed with Essential Tremor with a tremor measured in a healthy patient.

13. The method of claim 1, further comprising outputting an individual patient cognitive status.

14. The method of claim 1, further comprising outputting a group level cognitive status, wherein the group comprises the user and each member having a common attribute.

15. The method of claim 1, further comprising designating a class designation of cognitive impairment.

16. The method of claim 1, wherein the collection of multimodal data further comprises one or more questionnaire assessments or electronic health records, wherein, determining the second order feature uses data embedded in the one or more questionnaire assessments or electronic health records.

17. The method of claim 1, further comprising tracking the cognitive impairment status over time.

18. The method of claim 17, further comprising detecting changes in the cognitive impairment status based on said tracking.

19. A system comprising:
at least one input device;
a computing node coupled to the at least one input device and comprising a computer readable storage medium having program instructions embodied therewith, the program instructions executable by a processor of the computing node to cause the processor to perform a method comprising:
receiving a collection of multimodal data of a user interaction with the at least one input device;
processing the collection of multimodal data according to each modality within the collection of multimodal data;
deriving one or more first order features from the processed collection of multimodal data;
deriving one or more second order features from the processed collection of multimodal data;
creating a patient data model based on the first and second order features; and
applying a machine learning model to the patient data model to determine a cognitive impairment status.

20. A computer program product for determining a cognitive impairment status, the computer program product comprising a computer readable storage medium having program instructions embodied therewith, the program instructions executable by a processor to cause the processor to perform a method comprising:
receiving a collection of multimodal data of a user interaction with a computing device;
processing the collection of multimodal data according to each modality within the collection of multimodal data;
deriving one or more first order features from the processed collection of multimodal data;
deriving one or more second order features from the processed collection of multimodal data;
creating a patient data model based on the first and second order features; and
applying a machine learning model to the patient data model to determine a cognitive impairment status.
